(19) United States
(12) Patent Application Publication
(10) Pub. No.: US 2026/0132394 A1
(43) Pub. Date: May 14, 2026

(54) PENICILLIUM CYCLOPIUM STRAIN CAPABLE OF DEGRADING CROP STRAW FIBERS AND APPLICATION THEREOF

(71) Applicants: Shandong Agricultural University, Taian (CN); Shandong Normal University, Ji'nan (CN)

(72) Inventors: Xiuguo ZHANG, Taian (CN); Chunyuan ZHU, Taian (CN); Zhe MENG, Ji'nan (CN); Hui SHENG, Taian (CN); Yujiao WANG, Ji'nan (CN); Congcong AI, Ji'nan (CN); Cancan YANG, Taian (CN); Hao DONG, Taian (CN); Jiawang ZHANG, Taian (CN)

(73) Assignees: Shandong Agricultural University, Taian (CN); Shandong Normal University, Ji'nan (CN)

(21) Appl. No.: 19/383,810

(22) Filed: Nov. 10, 2025

(30) Foreign Application Priority Data
Nov. 11, 2024 (CN) 202411602497.4

(51) Int. Cl. C12N 9/42; C12N 1/14; C12P 21/02

(52) U.S. Cl. CPC C12N 9/2437; C12N 1/145; C12P 21/02; C12Y 302/01004

(57) ABSTRACT

A Penicillium cyclopium strain capable of degrading crop straw fibers efficiently and application thereof are provided. The provided induces mutagenesis of a Penicillium cyclopium strain MP-03 (2554-2) (CGMCC No. 41542) using a nitrosoguanidine method and obtains a mutant strain ΔMP-03 (455-1) (CGMCC No. 41541) with enhanced ability to degrade corn straw. By optimizing a nitrogen-to-carbon ratio of culture medium, culture time, temperature, and a pH range for high enzymatic activity, the mutant strain 455-1 significantly enhances the degradation of corn straw, and endoglucanase and exoglucanase activities in corn straw compared with the non-mutant strain 2554-2. The provided optimizes the conditions for scale-up culture of the mutant strain 455-1, and develops the organic-coupled cultivation technology that integrates a fermentation suspension of the mutant strain 455-1, corn straw and organic fertilizer, which improves the growth, root development and photosynthetic efficiency of greenhouse vegetable, and resistance to the soil-borne oomycete diseases.

### CROSS REFERENCE TO THE RELATED APPLICATIONS

This application is based upon and claims priority to Chinese Patent Application No. 202411602497.4, filed on Nov. 11, 2024, the entire contents of which are incorporated herein by reference.

### SEQUENCE LISTING

The instant application contains a Sequence Listing which has been submitted in XML format via EFS-Web and is hereby incorporated by reference in its entirety. Said XML copy is named GBDFHZ009_SequenceListing.xml, created on Oct. 30, 2025, and is 5,590 bytes in size.

### TECHNICAL FIELD

The present disclosure relates to the technical field of microorganisms and their applications, and in particular to a Penicillium cyclopium strain capable of degrading crop straw fibers efficiently and application thereof.

### BACKGROUND

Crop straw substrates are primarily composed of cell walls, which contain a large amount of carbohydrates, together with a small proportion of proteins and minerals. The carbohydrates mainly include cellulose, hemicellulose, and pectin polysaccharides. Straw returning to the field can increase soil organic matter, improve soil physicochemical properties, and reduce the loss of phosphorus and potassium nutrients contained in straw to some extent. However, crop straw is mainly composed of lignocellulose, which is difficult to be effectively decomposed by microorganisms under natural soil conditions, thereby significantly reducing its effect in improving soil fertility. In addition, straw degradation in soil requires a certain carbon-to-nitrogen ratio, which may easily lead to competitive absorption of nitrogen fertilizer between straw and crops, affecting vegetable growth. Therefore, by selecting or improving efficient straw-degrading microbial strains, it is feasible to develop novel microbial suspension-based technologies for organic coupling cultivation of degradation of crop straw substrates. At present, research on the screening and application of microbial strains for biological degradation of crop straw has been carried out, and several fungal strains capable of degrading crop straw have been identified. These fungal strains mainly include Pleurotus spp., Trichoderma spp., Penicillium spp., Aspergillus spp., and some bacterial consortia. Among them, the Aspergillus fumigatus strain XC6 achieved degradation rates of 72%, 93%, and 84% for straw cellulose, hemicellulose, and crop straw, respectively. Furthermore, from substrates such as soil, horse manure, and cow manure, thermophilic bacterial strains with strong cellulose decomposition ability, mesophilic actinomycete strains, and mesophilic fungal strains were isolated, and their effects on cellulase activity in rice straw substrates were respectively determined. Research on biological degradation of crop straw by Penicillium spp. is relatively limited; however, existing studies have shown that a few Penicillium spp. species exhibit strong capabilities in degrading lignocellulosic materials in crop straw. In addition, actinomycetes with strong degradation ability are widely distributed across diverse substrates, including soil, mature compost, horse manure, and rotten wood. Penicillium species has a simple structure, with unicellular conidia, which facilitates large-scale optimized cultivation and propagation. Based on the screening of Penicillium strains capable of degrading crop straw lignin with high efficiency, optimal nitrogen-carbon ratio, culture duration, temperature, and pH range of an enzyme-producing culture medium are determined to ensure that beneficial Penicillium strains for effective development and application are obtained.

### SUMMARY

An objective of the present disclosure is to provide a Penicillium cyclopium strain capable of degrading crop straw fibers efficiently and application thereof.

The present disclosure provides a Penicillium strain MP-03 (2554-2), which is isolated from corn straw and capable of improving cellulase activity in corn straw. This strain is identified as Penicillium cyclopium. The strain 2554-2 is induced to mutate using a nitrosoguanidine method, a mutant strain with enhanced ability to degrade the corn straw, designated as ΔMP-03 (455-1), was obtained. The strains 2554-2 and 455-1 have been deposited with China General Microbiological Culture Collection Center (CGMCC), at the deposit address of Microbiology, Chinese Academy of Sciences, No. 3, Yard 1, Beichen Road (W), Chaoyang District, Beijing, 100101, P.R.China on Oct. 12, 2024. The strain 2554-2 has an accession number of CGMCC NO. 41542, and the strain 455-1 has an accession number of CGMCC NO. 41541.

In a first aspect, the present disclosure provides a Penicillium cyclopium strain capable of degrading crop straw fibers efficiently, where the strain is Penicillium cyclopium 455-1, with an accession number of CGMCC No. 41541.

In a second aspect, the present disclosure provides a microbial agent containing the strain Penicillium cyclopium 455-1.

In a third aspect, the present disclosure provides application of the strain Penicillium cyclopium 455-1 in producing cellulase.

In a fourth aspect, the present disclosure provides application of Penicillium cyclopium 455-1 or a microbial agent containing the same in degrading corn straw.

In a fifth aspect, the present disclosure provides application of Penicillium cyclopium 455-1 or a microbial agent containing the same in improving the activities of endoglucanase and exoglucanase enzymes in crop straw.

The crop straw in the present disclosure includes, but is not limited to, corn straw.

In a sixth aspect, the present disclosure provides a method for degrading corn straw, where the corn straw is used as a raw material and inoculated with a fermentation suspension of Penicillium cyclopium 455-1, and degraded under the conditions of 20-40° C. and an initial pH of 6.0-8.0 (preferably 30° C., and an initial pH of 7.0).

Further, an appropriate amount of farmyard manure or bacterial fertilizer is added to the raw material.

Through the above technical solutions, the present disclosure has at least the following advantages and beneficial effects:

The present disclosure adopts a nitrosoguanidine method to induce mutagenesis of a Penicillium cyclopium strain MP-03 to obtain a mutant strain with enhanced ability to degrade the corn straw 455-1. By optimizing the nitrogen-to-carbon ratio of the culture medium, culture time, temperature, and pH range for high enzymatic activity, the mutant strain 455-1 significantly improves the degradation rate of corn straw and endoglucanase and exoglucanase activities in corn straw compared with the non-mutant strain MP-03. The present disclosure also optimizes the conditions for scale-up culture of the mutant strain 455-1, and develops an organic-coupled cultivation technology that integrates the fermentation suspension of the mutant strain 455-1, corn straw and organic fertilizer for greenhouse vegetable, which significantly improves the growth, root development, and photosynthetic efficiency of greenhouse tomatoes, peppers, and cucumbers, as well as the ability to inhibit the occurrence and harm of soil-borne oomycete diseases.

### BRIEF DESCRIPTION OF THE DRAWINGS

FIG. 1 is a comparison of morphological characteristics of strains ΔMP-03 (A) and MP-03 (B) of the present disclosure.

FIG. 2 is an agarose gel electrophoresis diagram of a complete ITS sequence of strains MP-03 and ΔMP-03 of the present disclosure.

FIG. 3 shows degradation effects of corn straw by strains ΔMP-03 and MP-03 according to a preferred embodiment of the present disclosure, where different letters indicate significant differences (P>0.05), and identical letters indicate no significant difference.

FIGS. 4A-4C show effects of temperature (FIG. 4A), initial pH (FIG. 4B), and different additives (FIG. 4C) on the degradation of corn straw by strains ΔMP-03 and MP-03 according to a preferred embodiment of the present disclosure. In FIG. 4C, different asterisks represent levels of significant difference in the degradation of corn straw **P<0.01, ***P<0.001. Each experiment was repeated at least three times.

FIG. 5 shows effects of strains ΔMP-03 and MP-03 on changes in the endoglucanase activity of corn straw according to a preferred embodiment of the present disclosure.

FIG. 6 shows effects of strains ΔMP-03 and MP-03 on changes in the exoglucanase activity of corn straw according to a preferred embodiment of the present disclosure.

### DETAILED DESCRIPTION OF THE EMBODIMENTS

The following embodiments are used for describing the present disclosure, but are not used for limiting the scope of the present disclosure. Unless otherwise specified, the technical means used in the embodiments are conventional means well known to those skilled in the art, and all raw materials used are commercially available.

In the following examples, data processing was performed using Excel of Microsoft Office 2003 for data analysis, tabulation, and plotting. Significance analysis was performed using SPSS 18.0 statistical software.

### Example 1: Identification, and Mutation of Penicillium cyclopium MP-03, and Optimization of Corn Straw Fermentation Technology

### 1. Materials and Methods

### 1.1 Test Materials

A plurality of Penicillium strains were isolated from autumn corn fields in Tai'an City, Shandong Province, China, and stored in a refrigerator at 4° C. A fungal genomic DNA Rapid Extraction Kit was purchased from Sangon Biotech (Shanghai) Co., Ltd.

Corn straw was obtained from corn fields in Tai'an City, Shandong Province, China during the autumn of 2022, and fresh mold-free corn straw was selected, washed, dried, crushed and passed through a 20-mesh sieve for subsequent use.

Solid slant culture medium: Potato dextrose agar culture medium containing 15 g agar, 20 g glucose, 200 g potato, and 1000 mL water. PD broth: potato dextrose (PD) broth containing 20 g glucose, 200 g potato, and 1000 mL water.

### 1.2 Enrichment, Isolation, and Screening of Corn Straw-Degrading Strains

Enrichment culture: 10 g of corn straw particle samples were added to 90 mL of sterile water and thoroughly shaken, and then allowed to stand for 10 min to obtain a mixture, 1 mL of a suspension was taken from the mixture and inoculated into 9 mL of the PD broth, a filter paper was added in the PD broth to serve as an indicator of visual degradation, and culture was performed statically at 30° C. When the filter paper was completely degraded, 1 mL of culture was transferred to 9 mL of fresh PD broth. This serial subculture process was repeated to eliminate cultures that lost the ability to degrade the filter paper, and the culture having strong decomposition capabilities for single bacterial isolation was retained.

Single-colony isolation: The enriched culture was serially diluted and evenly spread on PDA solid plates, which were then incubated in a 28° C. constant temperature incubator for 2-3 days. Single colonies with distinct morphologies were selected and cultured repeatedly on single plates. Single colonies were finally isolated and transferred on a PDA slant, and stored at 4° C. A plurality of isolated single colonies were inoculated into PD broth, and the complete disintegration of filter paper was used as an indicator to select candidate strains with potential corn straw-degrading ability.

### 1.3 Strain Identification Method

Observation of morphological characteristics: To observe the morphological characteristics of the strain, activated strains were streaked on PDA plates and incubated in a 28° C. incubator for 24 h. Sterilized glass slides were inserted into the plate at a 45° angle containing the growing strains, the strains were continuously incubated another 48 h, the slides were then gently removed to observe hyphae, conidia, and conidiophores under a microscope, and photomicrographs thereof were taken and arranged into plates.

Extraction of mycelial DNA: 250 mL Erlenmeyer flask containing 100 mL of PD broth was sterilized at 121° C. for 20 min and cooled to room temperature, and a Penicillium cyclopium MP-03 strain was inoculated and cultured at 28° C. with shaking at 150 r/min for 2 days. Mycelia cells were harvested by centrifugation, ground with liquid nitrogen, and DNA was extracted using the fungal genomic DNA rapid extraction kit and stored at −80° C. for subsequent use.

PCR amplification: Extracted DNA was pre-denatured at 94° C. for 5 min using an ITS forward primer (5′-GGAAGTAAAAGTCGTAACAAGG-3′, as shown in SEQ ID NO: 3) and a reverse primer (5′-TCCTCCGCTTATTGATATGC-3′, as shown in SEQ ID NO: 4) for the identification of Penicillium cyclopium, and Amplification was performed with 35 cycles of denaturation at 95° C. for 1 min, annealing at 55° C. for 1 min, and extension at 72° C. for 1 min

Purification PCR amplification products: PCR amplification products were detected by 5% agarose gel electrophoresis, gel fragments were excised and the target DNA fragments were purified using an agarose gel DNA recovery kit.

DNA sequencing and BLAST analysis: The gel-excision products were sent to Sangon Biotech (Shanghai) Co., Ltd. for sequencing. Sequencing results were analyzed using BLAST software to perform sequence homology analysis of the target gene.

Sequence analysis: Gene sequences for strains with high homology to the Penicillium strains under test were downloaded from GenBank for further comparison and analysis. The isolated MP-03 strain was identified by combining morphological characteristics with molecular DNA sequence comparison results.

### 1.4 Induction of Mutagenesis in MP-03 Strain by Nitrosoguanidine Method

Nitrosoguanidine (N-Methyl-N′-nitro-N-nitrosoguanidine, NTG) is an effective chemical mutagen, which exhibits a strong mutagenic effect at extremely low lethal concentrations, and is known as a supermutagen. The mutagenic effect of NTG primarily causes GC→AT transitions in DNA strands.

The mutagenesis procedures are as follows:

1) A loopful of the MP-03 strain from the slant culture was inoculated into a test tube containing 5 mL of PD broth and incubated at 25° C. with shaking for 48 h. Subsequently, 1.0 mL of the overnight broth was transferred to another tube containing 5 mL PD broth and incubated at 25° C. with shaking for 12 h. PDA culture medium was melted and cooled to 45° C., and approximately 20-25 mL was poured into each of 10-20 Petri dishes. After solidification, the plates were used for inoculation. An aliquot of 0.2 mL of the above Penicillium suspension was placed onto a surface of a single set of PDA plate, and the suspension was evenly spread over the entire surface using a sterile glass spreader.

2) A small amount of NTG crystals was placed near an edge of each PDA plate inoculated with the suspension, the plate was then carefully inverted and incubated in a 25° C. constant temperature incubator for 36 h. An inhibition zone appeared around the area where the NTG crystals were placed.

3) For the culture of putative mutant strains, a small amount of mycelia adjacent to the inhibition zone was picked and placed in one Erlenmeyer flask containing 20 mL of PD broth, and then shaken well to prepare a mycelial suspension. Meanwhile, a small amount of mycelia distant from the inhibition zone was picked and placed in another Erlenmeyer flask with 20 mL of PD broth to prepare a control mycelial suspension. The above two Erlenmeyer flasks were incubated at 25° C. with shaking overnight.

4) Plate spreading: 0.1 mL of each of the above two overnight mycelial suspensions was taken and spread onto an agar plate. Each overnight mycelial suspension was spread onto six plates, and three replicate plates were prepared for the control mycelial suspension. Plates after spreading were incubated in 25° C. constant temperature incubator for 48 h. In practice, the mycelial suspensions were diluted with sterile saline as appropriate to adjust concentrations, and a back of each plate was marked to distinguish treated and control plates.

5) A few drops of iodine solution was added to approximately 5-6 colonies formed after mutagenesis, a transparent zone appeared around the colonies, and diameters of the transparent zone and the colonies were measured separately to calculate a ratio thereof (HC ratio). By comparison with control plates, the mutagenic effect was analyzed. Colonies with a high HC ratio were selected and transferred to PDA slants or PDA plates for further observation and comparison of colonies, conidia, and conidiophore characteristics.

### 1.5 Enrichment and Fermentation of Corn Straw Mutant Strain 455-1 and Non-Mutant Strain MP-03

1) Preparation of fermentation suspensions of the strains 455-1 and MP-03: The mutant strain 455-1 and the non-mutant strain MP-03 were inoculated into 2,000 mL sterilized Erlenmeyer flasks containing 1,000 mL of PD broth, and cultured at 25° C. with shaking at 150 r/min for 3 days to obtain sufficient conidial suspensions, which was enriched to 2×10⁸ conidia/mL.

2) Fermentation on corn straw with the strains 455-1 and MP-03: Small bowls (with a diameter of 120 mm and a depth of 80 mm) were used as fermentation containers. Each bowl was filled with 20 g of powdered corn straw, and other substances were added according to the experimental design. The bowls were sterilized with high-temperature steam at 121° C. for 20 min and cooled, and a 10% (v/v) inoculation volume of the mycelial suspension was inoculated. The bowls were sealed in sterile plastic bags and incubated in a 25° C. incubator. Samples were collected once every 5 days during a 50-day fermentation cycle for determination of various enzyme activities and degradation of corn straw.

### 1.6 Determination of Degradation of Corn Straw by 455-1

The mutant strain suspension was inoculated at a 10% (v/v) inoculation volume into several Erlenmeyer flasks containing corn straw particle medium (50% corn straw particle, 36% corn cob particles, 6% wheat bran, 3% corn flour, 1% salt, 1% diammonium phosphate, 3% lime) and incubated statically in a 30° C. constant temperature incubator. The Erlenmeyer flasks were taken out from the incubator, and samples were collected on Days 1, 3, 5, 7, 9, and 11, respectively, to measure a weight loss of corn straw particles. Samples achieving peak degradation were analyzed for determining degradation rates of cellulose, hemicellulose and lignin. The non-mutant Penicillium suspension was used as a control.

### 1.7 Factors Affecting Degradation of Corn Straw by 455-1

1) Culture temperature: Fresh mutant Penicillium suspension was inoculated at a 10% (v/v) inoculation volume into corn straw particle medium and incubated statically at 20, 25, 30, 35, and 40° C., respectively. After 7 days, weight loss rates of corn straw particles were measured.

2) Initial pH: Corn straw particle media with initial pH values of 4, 5, 6, 7, 8, 9, and 10 were prepared. Fresh mutant Penicillium suspension was inoculated at a 10% (v/v) inoculation volume and incubated statically at 30° C. After 7 days, weight loss rates of corn straw particles were measured.

3) The corn straw particles for degradation consisted of the following components in a mass proportion: 50% corn straw (powdered or unpowdered), 8.2% peanut meal, 8.2% wheat bran, 9.7% farmyard manure (mainly including organic matter, nitrogen, phosphorus, potassium, and other nutrients, purchased from Shandong Lvbao Biotechnology Co., Ltd.), 6.8% bacterial fertilizer (Kingenta Soil Fertilizer No. 1), 5.2% compound fertilizer (Anyang Zhongsheng Fertilizer Co., content of nitrogen, phosphorus, potassium up to 45-55%), and 6.8% water. Mutant Penicillium mycelial suspension was added at a 5% inoculation volume (2×10⁸ conidia/mL), and a 0.1% vegetable virus vaccine (attenuated strain vaccine or bivalent small RNA vaccine; Nanjing Biotechnology Co., Ltd.) was added, then incubated statically at 30° C. After 7 days, weight loss rates of corn straw particles were measured. Non-mutant Penicillium suspension at a 10% (v/v) inoculation volume (2×10⁸ conidia/mL) was used as a control.

### 1.8 Determination of Weight Loss Rates of Corn Straw

The culture medium was filtered with filter paper, and the residual corn straw particles were repeatedly rinsed with a mixed solution of hydrochloric acid and nitric acid to eliminate mycelia cells. The residue was dried at 105° C. to a constant weight and then weighed. Weight loss rates of corn straw were calculated using a weight loss method. The calculation formula is as follows:

$$Weight loss rate of corn straw = ( {m}_{0} - m ) / {m}_{0} \times 100 \% \qquad Formula ( 1 )$$

In Formula (1): m₀ denotes a dry weight of residual corn straw particles in the control group (g), and m denotes a dry weight of residual corn straw particles in the treated group (g).

### 1.9 Determination of Cellulose, Hemicellulose and Lignin

Cellulose, hemicellulose and lignin contents of corn straw particles were determined by a Van Soest method. Samples were first treated with a neutral detergent solution to obtain a filtrate, and the filtrate was discarded. The residue was then treated with an acidic detergent solution; the resulting acidic detergent-soluble product was hemicellulose. The remaining acidic residue was then treated with 72% sulfuric acid, and the resulting soluble product was cellulose. The residue was dried and ashed, and a portion of loss during ashing was lignin.

Degradation rates of cellulose, hemicellulose and lignin were calculated using the following formula:

$$Degradation rate = ( {c}_{o} \times {m}_{o} - c \times m ) / ( {c}_{o} \times {m}_{o} ) \times 100 \% \qquad Formula ( 2 )$$

In Formula (2): cₒ denotes a content of cellulose, hemicellulose, or lignin in the control group (g); mₒ denotes a dry weight of residual corn straw particles in the control group (g), c denotes a content of cellulose, hemicellulose, or lignin in the treated group (g), and m denotes a dry weight of residual corn straw particles in the treated group (g). A non-mutant Penicillium suspension was used as the control group.

### 1.10 Determination of Cellulase Activity in Corn Straw

A glucose standard curve was plotted according to the experimental steps shown in Table 1. An absorbance of each sample in each test tube at 540 nm was measured. The glucose standard curve using glucose concentration was plotted, with glucose concentration on the x-axis and absorbance (OD) on the y-axis.

TABLE 1
Experimental Steps for Plotting Glucose Standard Curve

| Test Tube No. | 0 | 1 | 2 | 3 | 4 | 5 | 6 |
|---|---|---|---|---|---|---|---|
| 1 mg/mL Glucose standard | 0.0 | 0.4 | 0.6 | 0.8 | 1.0 | 1.2 | 1.4 |
| solution (mL) | | | | | | | |
| Distilled water (mL) | 2.0 | 1.6 | 1.4 | 1.2 | 1.0 | 0.8 | 0.6 |
| DNS reagent (mL) | | | | 2.0 | | | |

Boiled in a water bath for 5 min, and measured an absorbance at λ = 540 nm.

OD₅₄₀ value was obtained by determining cellulase activity and then converted to a corresponding glucose amount.

Mutant and non-mutant suspensions were inoculated at a 10% (v/v) inoculation volume into 50 mL of enzyme production medium and incubated statically at 30° C. Samples were collected on Days 0, 2, 4, 6, 8, and 10, respectively. The samples were centrifuged at 10,000 r/min for 5 min at 4° C. to collect supernatants, respectively, and the supernatants were collected as crude enzyme solutions for subsequent determination of cellulase activity.

The determination of cellulase activity was performed according to the method reported by Kazeem et al. (2017, Applied Biochemistry and Biotechnology, 182:1318-1340). The enzyme activity unit (U) was defined as an amount of enzyme required to release 1 μmol of reducing sugar in 1 min from the corn straw particle substrate under specific conditions. The reducing sugar was determined by a 3,5-dinitrosalicylic acid (DNS) colorimetric method according to method reported by Miller (1959, Analytical Chemistry, 31:426-428). A substrate for endoglucanase activity determination was a 1% carboxymethyl cellulose sodium (CMC-Na) solution prepared in 0.1 mol/L phosphate buffer (pH 6.0); a substrate for exoglucanase activity determination was a 1% cellulose solution prepared in the same buffer. 0.5 mL of crude enzyme solution was added to 1.5 mL of substrate solution and incubated in a 60° C. water bath for 30 min, 1.5 mL of DNS reagent was added immediately, followed by boiling in a water bath for 5 min to obtain a mixture, the mixture was cooled immediately and shaken thoroughly, and an absorbance was measured at 540 nm. An amount of reducing sugar released was determined using the absorbance against a glucose standard curve, and the corresponding enzyme activity was calculated. The non-mutant Penicillium suspension was used as a control group.

### 2. Results and Analysis

### 2.1 Screening of Candidate Fungal Strains for Degradation of Corn Straw

A fungal strain MP-03 having strong filter paper degradation ability was screened out from several isolated fungal strains and preliminarily identified as a candidate strain with the ability to degrade corn straw. The strain was subsequently induced to mutate by the nitrosoguanidine method; and a plurality of putative mutant strains were generated, and then transferred to PD culture medium and cultured at 28° C. to obtain mycelial suspensions. According to the filter paper degradation assay described above, the mycelial suspension with the strongest degradation ability was selected. The selected suspension was uniformly spread on a PDA solid plate and incubated at 28° C. for 2-3 days. Single colonies were picked and transferred to PDA slant tubes and cultured at 4° C. A candidate mutant strain with the strongest ability to degrade corn straw was selected and designated as ΔMP-03.

### 2.2 Morphological Characteristics of Non-Mutant and Mutant Fungal Strains

### (1) Morphological Identification

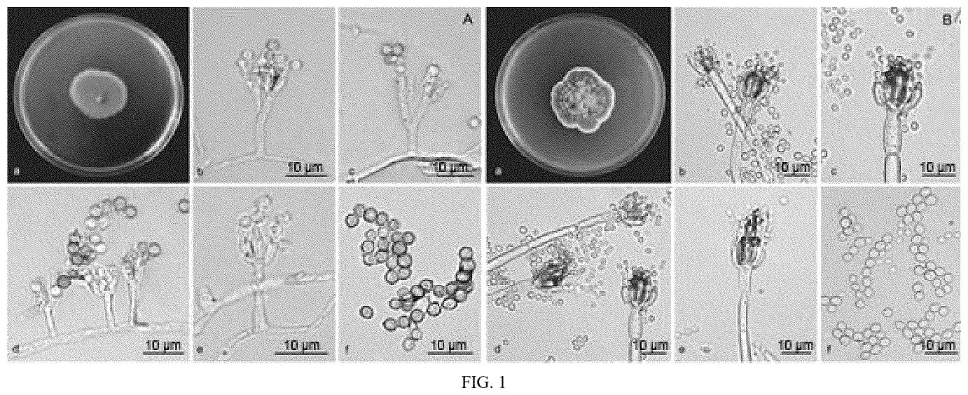

The selected strain MP-03 having the ability to degrade corn straw and the mutant strain ΔMP-03 having the strong degradation ability were cultured on PDA culture medium for 3-4 days. As shown in FIG. 1, a colony color of the MP-03 (A) changed from white to gray-green, the hyphae were septate and colorless; the conidiophores were septate and produced asymmetric broom-like structures and solitary secondary conidiophores. The tips of the conidiophores formed swollen vesicles, and the conidia were spherical, forming unbranched spore chains. The fungal strain was preliminarily identified as Penicillium cyclopium. A colony color of the mutant strain ΔMP-03 (B) changed from white to dark gray, the conidiophores were septate and produced dense clusters of reddish-brown broom-like conidiophores. The swollen tip regions were smaller, the branching of conidial chains was reduced, the conidia became smaller, and the number of conidia increased significantly.

### (2) Molecular Identification of MP-03 and ΔMP-03

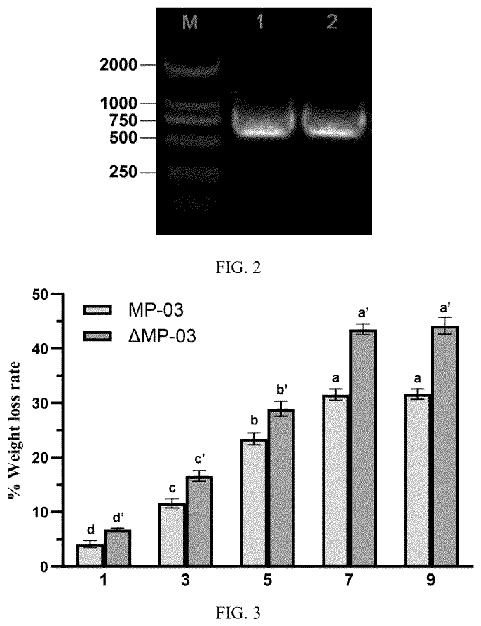

PCR products of the strains MP-03 and ΔMP-03 were analyzed by agarose gel electrophoresis. Genomic DNA from both mutant and non-mutant strains was amplified using ITS primers, resulting in single DNA fragments for each strain. A fragment size of each of the DNA fragments was generally consistent with a predicted size of Penicillium DNA fragment as determined from the web.expasy.org/protparam/, as shown in FIG. 2.

DNA sequencing results and homology comparison analysis: The DNA fragments of strains MP-03 and ΔMP-03 were excised and recovered from agarose gels, and subsequently sent to Sangon Biotech (Shanghai) Co., Ltd. for sequencing. Sequencing results were analyzed using BLAST software to perform homology comparison of the target gene sequences from MP-03 and ΔMP-03. By comparative analysis with Penicillium gene sequences in the GenBank database, the strains MP-03 and ΔMP-03 were both identified as Penicillium cyclopium. The ITS sequences of the strains MP-03 and ΔMP-03 are provided as SEQ ID NO: 1 and SEQ ID NO: 2, respectively.

### 2.3 Degradation Effect of ΔMP-03 and MP-03 on Corn Straw Effect

The differences in degradation of corn straw ability between the strains ΔMP-03 and MP-03 was examined by continuously sampling and measuring the weight loss rates of corn straw particles. As shown in FIG. 3, the weight loss rates of corn straw after treatment with both the strains ΔMP-03 and MP-03 increased over time, reaching a peak on Day 7. The strains ΔMP-03 and MP-03 caused the weight loss rates of corn straw to be 37.56% and 29.45%, respectively. Therefore, compared with the strain MP-03, the strain ΔMP-03 showed a significantly higher degradation rate of corn straw, and with prolonged treatment time, the degradation rate of the strain ΔMP-03 showed a stable increasing trend compared with the strain MP-03.

Corn straw samples treated with fermentation suspensions of the strains ΔMP-03 and MP-03 (2×10⁸ conidia/mL) were collected to determine the degradation of lignocellulosic components in the corn straw on Day 7 after being treated with the strains under test. As shown in Table 2, the strain ΔMP-03 fermentation suspension (2×10⁸ conidia/mL) significantly improved the ability to degrade cellulose, hemicellulose, and lignin compared with the strain MP-03. The degradation rates of cellulose, hemicellulose, and lignin by the strain ΔMP-03 increased by 23.47%, 21.51%, and 12.18%, respectively compared with the strain MP-03, indicating that the strain ΔMP-03 has excellent ability to degrade all three major components of corn straw. However, lignin is more difficult to be decomposed than the other two components, resulting in relatively stable content despite of rapid weight loss of corn straw. The results in FIG. 3 and Table 2 demonstrate that the MP-03 after mutation significantly reduced the weight of corn straw from Day 7 after the corn straw was treated, and substantially increased degradation of lignocellulosic components.

TABLE 2
Degradation Effect of Lignocellulosic Components of Corn Straw by ΔMP-03 and MP-03

| | | | Content (%) | | | | | | Degradation rate (%) | | |
|---|---|---|---|---|---|---|---|---|---|---|---|
| Time | Corn straw | | Cellulose | | Hemicellulose | | Lignin | | Cellulose | Hemicellulose | Lignin |
| (d) | mass (g) | | MP-03 | ΔMP-03 | MP-03 | ΔMP-03 | MP-03 | ΔMP-03 | ΔMP-03 | ΔMP-03 | ΔMP-03 |
| 0 | 7.12 ± | 7.02 ± | 28.45 ± | 27.5 ± | 14.45 ± | 13.76 ± | 10.20 ± | 9.24 ± | — | — | — |
| | 0.01a | 0.23a | 0.23a | 0.15a | 0.15a | 0.47b | 0.23a | 0.34a | | | |
| 7 | 5.46 ± | 3.24 ± | 24.56 ± | 18.04 ± | 11.02 ± | 8.65 ± | 8.16 ± | 6.85 ± | 23.47 ± | 21.51 ± 0.35 | 12.18 ± |
| | 0.14b | 0.15c | 0.65b | 0.5c | 0.56b | 0.34c | 0.53b | 0.54c | 0.56 | | 0.86 |

Note:
In the table, identical letters in the same column indicate no significant difference, while different letters indicate significant differences (P > 0.05).

### 2.4 Determination of Effects of ΔMP-03 and MP-03 on Degradation of Corn Straw Under Different Culture Conditions

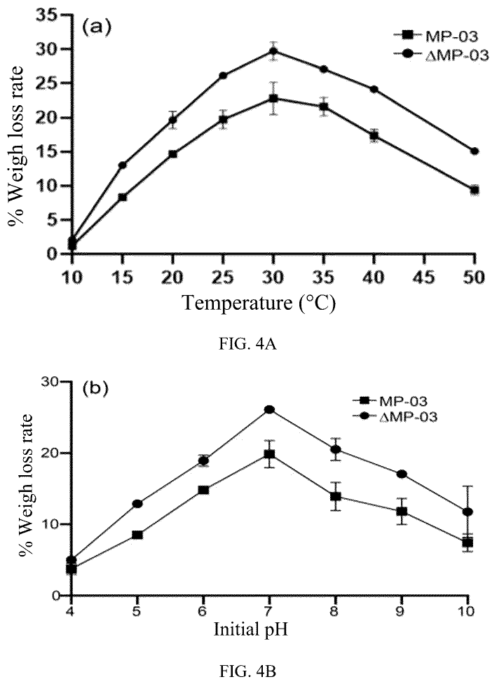

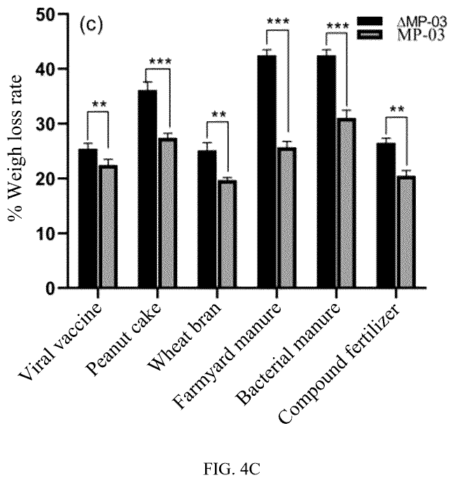

The effects of the ΔMP-03 and MP-03 fermentation suspensions (2×10⁸ conidia/mL) on degradation of corn straw under different temperatures were determined on Day 7 after treatment. Corn straw samples treated with the ΔMP-03 and MP-03 fermentation suspensions (2×10⁸ conidia/mL) were collected on Day 7 after treatment. FIG. 4A shows the effects of ΔMP-03 and MP-03 fermentation suspensions (2×10⁸ conidia/mL) on the degradation of corn straw at different conditions. As the temperature increased, the weight loss rates of corn straw increased initially and then decreased. At 30° C., the ΔMP-03 and MP-03 fermentation suspensions (2×10⁸ conidia/mL) showed the best degradation rates of corn straw, and the degradation rate of the ΔMP-03 was significantly higher than that of the MP-03, achieving 40.35% and 30.65%, respectively. When the treatment temperature was increased to 40° C., the ΔMP-03 and MP-03 fermentation suspensions both demonstrated high degradation rates of corn straw, with the ΔMP-03 outperforming the MP-03, and the weight loss rates of corn straw reached 27.35% and 20.45%, respectively. These results suggest that in autumn greenhouse vegetable cultivation, the addition of Penicillium cyclopium ΔMP-03 under suitable soil temperature conditions using the organic-coupled cultivation technology can effectively degrade corn straw by the ΔMP-03, and the degradation of corn straw can help increase soil temperature by 4-5° C., further promoting degradation of corn straw and effectively leveraging the benefits of organic-coupled cultivation technology.

The effects of the ΔMP-03 and MP-03 fermentation suspensions (2×10⁸ conidia/mL) on degradation of corn straw under different initial pH conditions were determined on Day 7 after treatment. Corn straw samples treated with the ΔMP-03 and MP-03 fermentation suspensions were collected under different initial pH conditions on Day 7 after treatment. FIG. 4B shows the effects of the ΔMP-03 and MP-03 fermentation suspensions on the degradation of corn straw under different initial pH conditions, the results indicated that the ΔMP-03 exhibited a higher degradation rate of corn straw than the MP-03 under all pH conditions. When an initial pH was 7.0, the weight loss rates of corn straw caused by the ΔMP-03 and MP-03 fermentation suspensions were 34.56% and 29.95%, respectively. However, extremely low or high pH significantly reduced the degradation rates of corn straw by the ΔMP-03 and MP-03 fermentation suspensions. When the pH was below 6.0 or above 8.0, the degradation rates of corn straw by the ΔMP-03 and MP-03 fermentation suspensions declined sharply. When the pH was increased to 10.0, the degradation rates of corn straw by the ΔMP-03 and MP-03 fermentation suspensions were the lowest. Therefore, the initial PH value should be controlled at 6.0-8.0. Both the ΔMP-03 and MP-03 fermentation suspensions exhibited the high degradation rates of corn straw, ensuring that the ΔMP-03 fermentation suspension consistently exhibited significantly higher degradation rate of corn straw than the MP-03 fermentation suspension.

The effects of the ΔMP-03 and MP-03 fermentation suspensions (2×10⁸ conidia/mL) containing various additives on degradation of corn straw were determined on Day 7 after treatment. Corn straw samples treated with the ΔMP-03 and MP-03 fermentation suspensions containing various additives were collected on Day 7 after treatment. FIG. 4C shows the effects of the ΔMP-03 and MP-03 fermentation suspensions containing various additives on degradation of corn straw. The results indicated that, by adding of virus vaccine, peanut cake, wheat bran, farmyard manure, bacterial fertilizer, or compound fertilizer, the ΔMP-03 fermentation suspension resulted in higher degradation rates of corn straw those that of the MP-03 fermentation suspension. Furthermore, farmyard manure and bacterial fertilizer induced the highest degradation rate of corn straw by the ΔMP-03 and the MP-03 fermentation suspensions, with weight loss rates of corn straw both approaching 30.54%. An order of degradation rates of corn straw by the ΔMP-03 and MP-03 fermentation suspensions containing various additives was: farmyard manure≥bacterial fertilizer>peanut cake>compound fertilizer>wheat bran>virus vaccine.

The above research results demonstrate that under different culture conditions, the ΔMP-03 fermentation suspension exhibits higher degradation rate of corn straw than the MP-03. The ΔMP-03 and MP-03 fermentation suspensions exhibited the best degradation rates of corn straw at a temperature of 30° C., an initial pH of 7.0, and with the addition of farmyard manure or bacterial fertilizer. The ΔMP-03 fermentation suspension consistently outperforming the MP-03 fermentation suspension.

### 2.5 Effects of ΔMP-03 and MP-03 Fermentation Suspensions on Changes in Cellulase Activity of Corn Straw

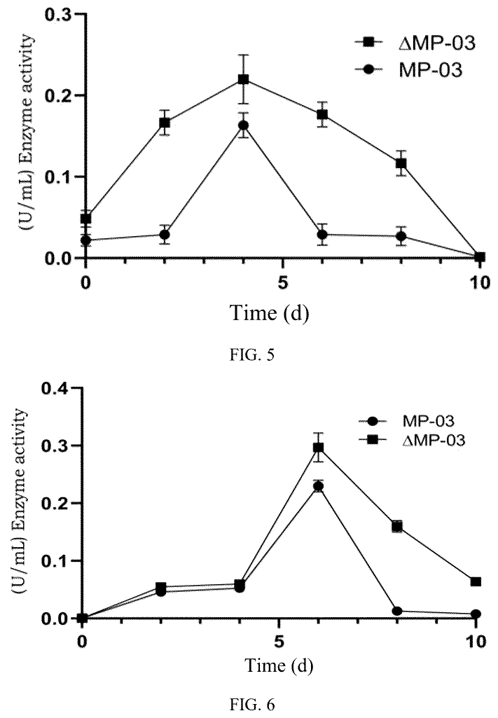

Endoglucanase primarily acts on amorphous regions of cellulose, cleaving long cellulose chains into shorter chains. Exoglucanase mainly acts on crystalline regions of cellulose, hydrolyzing cellulose from the ends to produce cellobiose. Corn straw was treated with the ΔMP-03 and MP-03 fermentation suspensions (2×10⁸ conidia/mL), and the activities of endoglucanase and exoglucanase in corn straw were measured at Days 0, 2, 4, 6, 8, and 10 after the treatment. FIG. 5 shows that the endoglucanase activity of corn straw treated with the ΔMP-03 and MP-03 fermentation suspensions reached peak on Day 4 (0.2 U/mL), indicating that the selected strain can effectively activate endoglucanase activity in corn straw on Day 4, which is conducive to the effective function of exoglucanase. Furthermore, across all treatment times, the ΔMP-03 fermentation suspension consistently showed higher endoglucanase activity than the MP-03 fermentation suspension, demonstrating that the ΔMP-03 fermentation suspension has a stronger ability to activate endoglucanase in corn straw, more effectively degrading the amorphous regions of cellulose and providing sufficient substrate for exoglucanase to act, thereby accelerating the conversion of cellulose into cellobiose. Furthermore, the exoglucanase activities of corn straw treated with the ΔMP-03 and the MP-03 were measured on Day 0, 2, 4, 6, 8, and 10 days. FIG. 6 shows that the exoglucanase activity of corn straw was the highest after treatment with the ΔMP-03 and the MP-03 on Day 6 after the treatment, and the ΔMP-03 consistently exhibited higher exoglucanase activity than the MP-03 across all treatment times. These results indicate that the ΔMP-03 exhibits stronger activation of both endoglucanase and exoglucanase activities in corn straw that that of the MP-03, increasing cellobiose production and decomposition and utilization of corn straw. Consequently, the organic matter content of soil under organic-coupled cultivation with corn straw is effectively increased, which is beneficial for improving the organic nutrient content and composition of soil in greenhouse vegetable cultivation.

Although the present disclosure has been described in detail with general descriptions and specific embodiments, various modifications or improvements can be made based on the present disclosure, which would be obvious to those skilled in the art. Therefore, all such modifications or improvements made without departing from the spirit of the present disclosure should fall within the scope of protection claimed by the present disclosure.

What is claimed is:

1. A Penicillium cyclopium strain capable of degrading crop straw fibers, wherein the strain is Penicillium cyclopium 455-1, with an accession number of CGMCC No. 41541.

2. A microbial agent comprising the Penicillium cyclopium strain according to claim 1.

3. An application of the Penicillium cyclopium strain according to claim 1 in producing cellulase.

4. An application of the Penicillium cyclopium strain according to claim 1 or a microbial agent in degrading a crop straw, wherein the microbial agent comprises the Penicillium cyclopium strain.

5. An application of the Penicillium cyclopium strain according to claim 1 or a microbial agent in improving activities of endoglucanase and exoglucanase enzymes in a crop straw, wherein the microbial agent comprises the Penicillium cyclopium strain.

6. The application according to claim 4, wherein the crop straw is a corn straw.

7. A method for degrading a corn straw, comprising using the corn straw as a raw material and inoculating the corn straw with a fermentation suspension of the Penicillium cyclopium strain according to claim 1, and degrading the corn straw under conditions of 20-40° C. and an initial pH of 6.0-8.0.

8. The method according to claim 7, wherein an appropriate amount of a farmyard manure or a bacterial fertilizer is added to the raw material.

9. The method according to claim 7, wherein the corn straw is degraded under conditions of 30° C. and an initial pH of 7.0.

10. The application according to claim 5, wherein the crop straw is a corn straw.

11. The method according to claim 8, wherein the corn straw is degraded under conditions of 30° C. and an initial pH of 7.0.


SEQUENCE LISTING (4 sequences)

SEQ ID NO: 1 - DNA, 570 bp, Penicillium cyclopium
  source 1..570: mol_type = genomic DNA
aaggtttccgtaggtgaacctgcggaaggatcattactgagtgagggccctctgggtccaacctcccacccgtgtttattgtaccttgttgcttcggtgcgcccgcctcacggccgccggggggcttctgcccccgggcccgcgcgcaccggagacaccattgaactctgtctgaagattgcagtctgagcataaactaaataagttaaaactttcaacaacggatctcttggttccggcatcgatgaagaacgcagcgaaatgcgataactaatgtgaattgcagaattcagtgaatcatcgagtctttgaacgcacattgcgccccctggtattccggggggcatgcctgtccgagcgtcattgctgccctcaagcacggcttgtgtgttgggctccgtccccccggggacgggcccgaaaggcagcggcggcaccgagtccggtcctcgagcgtatggggctttgtcacccgctctgtaggcccggccggcgccagccgacaacccatcatccttttcaggttgacctcggatcaggtagggatacccgctgaacttaagcatatca

SEQ ID NO: 2 - DNA, 560 bp, Penicillium cyclopium
  source 1..560: mol_type = genomic DNA
aaggtttccgtaggtgaacctgcggaaggatcattactgagtgagggccctctgggtccacctcccacccgtgtttattgtaccttgttgcttcggcgggcccgcctttatggccgccggggggcttacgctcccgggcccgcgcccgccgaagacaccctcgaactctgtctgaagattgtagtctgagtgaaaatataaattatttaaaactttcaacaacggatctcttggttccggcatcgatgaagaacgcagcgaaatgcgatacgtaatgtgaattgcagaattcagtgaatcatcgagtctttgaacgcacattgcgccccctggtattccggggggcatgcctgtccgagcgtcattgctgccctcaagcacggcttgtgtgttgggccccgtcctccgatcccgggggacgggcccgaaaggcagcggcggcaccgcgtccggtcctcgagcgtatggggctttgtcacccgctctgtaggcccggccggcgcttgccgatcaaccaaaattttttccaggttgacctcggatcaggtagggatacccgc

SEQ ID NO: 3 - DNA, 22 bp, synthetic construct
  source 1..22: mol_type = other DNA
ggaagtaaaagtcgtaacaagg

SEQ ID NO: 4 - DNA, 20 bp, synthetic construct
  source 1..20: mol_type = other DNA
tcctccgcttattgatatgc